(19) United States
(12) Patent Application Publication
(10) Pub. No.: US 2012/0070392 A1
(43) Pub. Date: Mar. 22, 2012

(54) COMPOSITION FOR INHIBITING ERYTHEMA CAUSED BY ULTRAVIOLET RADIATION CONTAINING A DIPEPTIDE AS ACTIVE INGREDIENT

(76) Inventors: Hyun-Kyung Lee, Seoul (KR); Hye-Ryung Choi, Seoul (KR); Youn-A Kang, Seoul (KR)

(21) Appl. No.: 13/258,935

(22) PCT Filed: Mar. 30, 2010
(86) PCT No.: PCT/KR10/01923

§ 371 Date: Oct. 21, 2011

(30) Foreign Application Priority Data
Mar. 31, 2009 (KR) 10-2009-0027344

(51) Int. Cl. A61K 8/64; A61Q 17/04; A61K 38/05

(52) U.S. Cl. 424/59; 514/18.6

(57) ABSTRACT

The present invention relates to a composition for inhibiting erythema caused by ultraviolet radiation, wherein said composition contains a specific dipeptide, selected from 400 dipeptides, as an active ingredient. The composition of the present invention inhibit the production of prostaglandin E₂(PEG₂), thereby inhibiting erythema caused by ultraviolet radiation.

### TECHNICAL FIELD

The present invention relates to a composition for suppressing UV light-induced erythema comprising a dipeptide as an active ingredient. More particularly, the present invention relates to a composition that functions to inhibit the production of PGE₂(prostaglandin E₂), an inflammation mediator whose biosynthesis is promoted by UV light, thereby controlling both UV-induced erythema and inflammation.

### BACKGROUND ART

UV light induces skin reactions such as erythema, sunburn, pigmentation, skin cancer, etc. Such UV-induced erythema consists of an immediate and a delayed phase. Mediating the dilation of vessels and the increase of vascular permeability, prostaglandin(hereinafter referred to as “PG”), histamine, serotonin, and interleukin, all of which are produced by keratinocytes, are known to be involved in the skin reactions. This invention is directed to the inhibition of prostaglandin activity.

Prostaglandins(PGs) are present in nearly every tissue or organs and are produced by almost all nucleated cells except for lymphocytes. PGs, derived from fatty acid, act on platelets, endothelium, mast cells and so on, and the cyclooxygenase-1(COX-1) or cyclooxygenase-2(COX-2) pathway is responsible for the biosynthesis of PGs.

PGs are structurally based on prostanoic acid and can be divided into PGA, PGB, PGC, PGD, PGE, PGF, PGG, PGH, PGI according to variation in the ring structure of prostanoic acid. Numbers can be applied following the number of double bond of side chain. PG performs various functions. For example, PGI₂ inhibits platelet aggregation; PGF₂ causes vascular constriction; PGE₂ causes vascular dilatation and bronchial dilatation and inhibits gastric acid secretion; and TXA2 promotes platelet aggregation, etc.

Among PGs, PGE₂ is known to be involved in skin inflammation and erythema. In light of the observation that a genetically modified mouse which lacks PGE₂ receptors such as EP2 and EP4 shows a much lower inflammatory response to UV irradiation, PGE2 is understood to play an important role in inducing UV-induced inflammation and erythema. Thus, if the production of PGE₂ is inhibited, it will be possible to control UV-induced erythema and inflammation, which led to the present invention.

There are patents that are concerned with the anti-inflammatory action of peptides.

Korean Patent Laid-Open Publication No. 2007-0116134 discloses a personal care composition comprising a dipeptide with threonine at the C-terminus plus a carrier or an injectable solution. This composition is used only to control keratinization and there are very few kinds of dipeptides that are available.

Japanese Patent Laid-Open Publication No. 1997-124434 discloses a topical agent comprising urea and a peptide. This topical agent is described as a good moisturizer which controls skin pH and ammonia production, but is not related with UV protection effects.

U.S. Pat. No. 3,965,260 addresses the use of a peptide as a therapeutic agent for rheumatic diseases.

### DISCLOSURE

### Technical Problem

It is an object of the present invention to provide a composition for suppressing UV-induced skin reactions, especially erythema, by inhibiting PGE₂ production, comprising a dipeptide as an active ingredient.

### Technical Solution

In accordance with an aspect thereof, the present invention provides a composition for suppressing UV-induced erythema, comprising as an active ingredient a dipeptide selected from the group consisting of SI(serine-isoleucine), YK(tyrosine-lysine), VC(valine-cysteine), MD(methionine-aspartic acid), WF(tryptophan-phenylalanine), NR(asparagine-arginine), DP(aspartic acid-proline), YE(tyrosine-glutamic acid), RM(arginine-methionine), QT(glutamine-threonine), LH(leucine-histidine), SP(serine-proline), IT(isoleucine-threonine), FW(phenylalanine-tryptophan), AP(alanine-proline), WP(tryptophan-proline), AN(alanine-asparagine), AE(alanine-glutamic acid), DT(aspartic acid-threonine), PT(proline-threonine), WD(tryptophan-aspartic acid), NA(asparagine-alanine), WY(tryptophan-tyrosine), AS(alanine-serine), DG(aspartic acid-glycine), SY(serine-tyrosine), NY(asparagine-tyrosine), LE(leucine-glutamic acid), VH(valine-histidine), TI(threonine-isoleucine), LR(leucine-arginine), FN(phenylalanine-asparagine), YR(tyrosine-arginine), NE(asparagine-glutamic acid), SL(serine-leucine), GL(glycine-leucine), MY(methionine-tyrosine), AA(alanine-alanine), IQ(isoleucine-glutamine), HH(histidine-histidine), NP(asparagine-proline), RP(arginine-proline), SD(serine-aspartic acid), AK(alanine-lysine), TD(threonine-aspartic acid), KY(lysine-tyrosine), LF(leucine-phenylalanine), IK(isoleucine-lysine), AF(alanine-phenylalanine), RS(arginine-serine), FK(phenylalanine-lysine), ST(serine-threonine), EP(glutamic acid-proline), WL(tryptophan-leucine), NC(asparagine-cysteine), RC(arginine-cysteine), SQ(serine-glutamine), WV(tryptophan-valine), SM(serine-methionine), PA(proline-alanine), YA(tyrosine-alanine), HY(histidine-tyrosine), YD(tyrosine-aspartic acid), RD(arginine-aspartic acid), KD(lysine-aspartic acid), DD(aspartic acid-aspartic acid), YI(tyrosine-isoleucine), AR(alanine-arginine), AC(alanine-cysteine), YQ(tyrosine-glutamine), VF(valine-phenylalanine), LC(leucine-cysteine), KT(lysine-threonine), QW(glutamine-tryptophan), RK(arginine-lysine), NL(asparagine-leucine), GQ(glycine-glutamine), GI(glycine-isoleucine), HK(histidine-lysine), DH(aspartic acid-histidine), VD(valine-aspartic acid), VY(valine-tyrosine), MT(methionine-threonine), QK(glutamine-lysine), TK(threonine-lysine), TV(threonine-valine), QQ(glutamine-glutamine), WC(tryptophan-cysteine), YY(tyrosine-tyrosine), ET(glutamic acid-threonine), QF(glutamine-phenylalanine), RN(arginine-asparagine), TR(threonine-arginine), AQ(alanine-glutamine), GD(glycine-aspartic acid), WA(tryptophan-alanine), IH(isoleucine-histidine), WW(tryptophan-tryptophan), QN(glutamine-asparagine), VI(valine-isoleucine), AD(alanine-aspartic acid), VN(valine-asparagine), CW(cysteine-tryptophan), EI(glutamic acid-isoleucine), SN(serine-asparagine), WN(tryptophan-asparagine), TG(threonine-glycine), SA(serine-alanine), GA(glycine-alanine), PW(proline-tryptophan), PQ(proline-glutamine), IG(isoleucine-glycine), SH(serine-histidine), VR(valine-arginine), KM(lysine-methionine), VK(valine-lysine), GF(glycine-phenylalanine), NV(asparagine-valine), RG(arginine-glycine), HW(histidine-tryptophan), VQ(valine-glutamine), CM(cysteine-methionine), KC(lysine-cysteine), WQ(tryptophan-glutamine), PD(proline-aspartic acid), NQ(asparagine-glutamine), AY(alanine-tyrosine), GS(glycine-serine), TW(threonine-tryptophan), EF(glutamic acid-phenylalanine), EG(glutamic acid-glycine), IV(isoleucine-valine), QY(glutamine-tyrosine), HL(histidine-leucine), SK(serine-lysine), ID(isoleucine-aspartic acid), TS(threonine-serine), GW(glycine-tryptophan), PI(proline-isoleucine), NH(asparagine-histidine), PY(proline-tyrosine), TP(threonine-proline), ED(glutamic acid-aspartic acid), DV(aspartic acid-valine), DA(aspartic acid-alanine), NT(asparagine-threonine), DQ(aspartic acid-glutamine), PF(proline-phenylalanine), SV(serine-valine), DS(aspartic acid-serine), GY(glycine-tyrosine), WR(tryptophan-arginine), NN(asparagine-asparagine), FA(phenylalanine-alanine), TQ(threonine-glutamine), IS(isoleucine-serine), VE(valine-glutamic acid), FR(phenylalanine-arginine), QR(glutamine-arginine), RL(arginine-leucine), TM(threonine-methionine), CN(cysteine-asparagine), FL(phenylalanine-leucine), TE(threonine-glutamic acid), DI(aspartic acid-isoleucine), RW(arginine-tryptophan), RT(arginine-threonine), MQ(methionine-glutamine), VP(valine-praline), WK(tryptophan-lysine), QH(glutamine-histidine), SE(serine-glutamic acid), AG(alanine-glycine), HS(histidine-serine), HP(histidine-proline), RE(arginine-glutamic acid), QS(glutamine-serine), SG(serine-glycine), AL(alanine-leucine), VA(valine-alanine), YL(tyrosine-leucine), YT(tyrosine-threonine), SR(serine-arginine), WS(tryptophan-serine), RY(arginine-tyrosine), FI(phenylalanine-isoleucine), VG(valine-glycine), LV(leucine-valine), WM(tryptophan-methionine), DW(aspartic acid-tryptophan), DC(aspartic acid-cysteine), FG(phenylalanine-glycine), HF(histidine-phenylalanine), PG(proline-glycine), CE(cysteine-glutamic acid), VV(valine-valine), DN(aspartic acid-asparagine), FH(phenylalanine-histidine), GH(glycine-histidine), MW(methionine-tryptophan), IP(isoleucine-proline), AI(alanine-isoleucine), ES(glutamic acid-serine), EA(glutamic acid-alanine), QV(glutamine-valine), MR(methionine-arginine), VL(valine-leucine), PK(proline-lysine), DM(aspartic acid-methionine), GG(glycine-glycine), YF(tyrosine-phenylalanine), EE(glutamic acid-glutamic acid), TF(threonine-phenylalanine), ER(glutamic acid-arginine), CS(cysteine-serine), IA(isoleucine-alanine), YM(tyrosine-methionine), IM(isoleucine-methionine), EV(glutamic acid-valine), SS(serine-serine), AH(alanine-histidine), EH(glutamic acid-histidine), NF(asparagine-phenylalanine), EM(glutamic acid-methionine), HA(histidine-alanine), RR(arginine-arginine), IY(isoleucine-tyrosine), SC(serine-cysteine), GK(glycine-lysine), PS(proline-serine), EY(glutamic acid-tyrosine), LK(leucine-lysine), CQ(cysteine-glutamine), KV(lysine-valine), WE(tryptophan-glutamic acid), HG(histidine-glycine), EK(glutamic acid-lysine), FF(phenylalanine-phenylalanine), FM(phenylalanine-methionine), DK(aspartic acid-lysine), LT(leucine-threonine), FD(phenylalanine-aspartic acid), DF(aspartic acid-phenylalanine), FY(phenylalanine-tyrosine), QD(glutamine-aspartic acid), LN(leucine-asparagine), KW(lysine-tryptophan), NS(asparagine-serine), PH(proline-histidine), WG(tryptophan-glycine), EL(glutamic acid-leucine), EQ(glutamic acid-glutamine), LA(leucine-alanine), NG(asparagine-glycine), NM(asparagine-methionine), WH(tryptophan-histidine), DE(aspartic acid-glutamic acid), DL(aspartic acid-leucine), AV(alanine-valine), PN(proline-asparagine), PR(proline-arginine) and a combination thereof.

In one preferred embodiment, the amount of the dipeptide that may be used is from 0.001˜30 wt % based on the total weight of the composition.

In another preferred embodiment, the composition may further comprise a pharmaceutically acceptable carrier or excipient.

In a further preferred embodiment, the composition may be in the form of a cosmetic such as a skin lotion, a lotion, a cream, a foundation, an essence, a gel, a pack, a foam cleanser, and a soap, or a medicament such as a topical ointment.

### ADVANTAGEOUS EFFECTS

Functioning to inhibit PGE₂ production, the composition of the present invention can suppress UV-induced erythema and inflammation. In addition, the composition comprising a dipeptide is safe to the body.

### Mode for Invention

Generally, the synthesis of peptides is expensive. Especially, it is difficult for the skin to absorb long peptides because of their high molecular weight. In the present invention, dipeptides, which are not expensive to synthesize and which can avoid the absorption problem was examined for ability to inhibit PEG₂ production was investigated. For this purpose, a library of 400 dipeptides was prepared and were screened for their PGE₂ inhibiting effects on cultured normal human keratinocytes irradiated with UV light.

As will be explained later, all 400 of the dipeptides were not able to control UV-induced erythema and inflammation, but specific kinds of dipeptides exhibit the inhibitory effect, as summarized in Table 2, below.

The content of the dipeptide in the composition of the present invention may vary depending on the purpose and method of use, and preferably ranges from 0.001 to 30 wt % based on the total weight of the composition.

The composition of the present invention may further comprise a pharmaceutically acceptable carrier or excipient. Representative carriers or excipients include water, dextrin and physiological saline.

In addition, the composition suppressive of UV-induced skin reactions in accordance with the present invention may be in the form of a cosmetic, a cleanser or a medicament. For a cosmetic, the composition of the present invention may be formulated into an astringent, a skin lotion, a nutrient cream, a massage cream, an essence, a pack, a soap, a shampoo, a skin patch or a skin gel. For use as a cleansing composition, the composition of the present invention may be formulated into a face wash or a bath agent. Examples of the medicaments into which the composition of the present invention can be formulated include plasters, granules, lotions, powders, syrups, ointments, emulsions, suspensions, tablets and injections.

Different amounts of the composition of the present invention may be used depending on the purpose and application site. For example, the composition may be used at a daily dose of from 0.001 to 100 mg/mL one to three times a day.

A better understanding of the present invention may be obtained through the following examples which are set forth to illustrate, but are not to be construed as limiting the present invention.

### INDUCTION OF PGE ₂ PRODUCTION BY UVB

Human keratinocytes were seeded at a density of 1×10⁵ cells/well to 6-well plates containing 2 mL of KGM(keratinocyte growth medium, Clonetics, San Diego, Calif., USA) per well and cultured overnight. Then, the medium was replaced with KBM(keratinocyte basal medium) supplemented with 0.1% BSA(bovine serum albumin) and the cells were cultured for an additional 24 hours. Subsequently, the cells were pre-treated for an additional 24 hours with 2 mL of the same medium containing 10 μg/ml of each of the dipeptides listed in Table 1. NS-398(N194, Sigma, St. Louis, Mo., USA), used as a positive control, was treated with 5 μM for one hour before UVB irradiation. The pre-treated medium was transferred to new 24 well plates and 1 mL of DPBS(Dulbecco's phosphate buffered saline) was added to the cells in each well. During UV irradiation, the transferred medium was stored in a cell incubator. UV B(100 mJ/cm²) was irradiated into the cells to induce the production of PGE₂. At this time, the control was covered with aluminum foil. After the removal of DPBS, the transferred medium was returned to each well. The cells were further cultured for 24 hours, after which 1 mL of the medium was transferred to microcentrifuge tubes. To each well containing the cell was added 100 μl MTT(3-(4, 5-dimetylthiazol-2-yl)-2,5-diphenyltetrazoliumbromide), followed by incubation for 3˜4 hours. The supernatants were then removed and the formazan crystals were solubilized in 1 ml of DMSO(dimethylsulfoxide). One hundred μL of the solution was transferred to each well of 96-well plates and an optical density was determined at 540 nm using an ELISA(enzyme-linked immunosorbent assay) reader. Meanwhile, the medium transferred to the microtubes was centrifuged at a speed of 12,00˜15,000 rpm for 5 min under freezing conditions and the supernatants were stored in new tubes in a freezer until the PGE₂ assay.

### PGE₂ Assay

The concentration of PGE₂ was determined using a PGE₂ Assay Kit(KGE004, R&D Systems, Inc., Minneapolis, Minn., USA) according to the manufacturer's instruction. PGE₂ standard solutions with concentrations of 2500, 1250, 625, 312, 156, 78, and 39 pg/mL were prepared. To each well of the 96-well plates provided in the kit were added 100 μL of the medium supernatant and 100 μL of the PGE₂ standard solutions. Under a light-tight condition, 50 μL of a primary PGE₂ antibody solution was added, and reacted with a PGE₂ conjugate at room temperature for 2 hours with stirring. After removal of the reaction, the wells were washed five times with 400 μL of wash buffer. To each well was added 200 μL of a substrate solution, followed by incubation for 20˜30 min. The reaction was terminated with 50 μL of a stop solution per well before determining absorbance at 450 nm on an ELISA reader. The PGE₂ concentration of each sample was calculated from the standard graph and calibrated with the absorbance obtained in the MTT assay to determine their inhibitory activity against PGE₂ production. When PEG₂ production was inhibited by 10% or more, the dipeptides were regarded as effective while an efficiency of 0% or lower was expressed as zero. The results are summarized in Table 1, below.

TABLE 1
% Inhibition against PGE₂ Production

| | % | | % | | % | | % | | % |
|---|---|---|---|---|---|---|---|---|---|
| Sample | Inhibition | Sample | Inhibition | Sample | Inhibition | Sample | Inhibition | Sample | Inhibition |
| SI | 50.3 | PY | 24.8 | TR | 53.0 | DH | 59.2 | VW | 0.0 |
| YE | 74.2 | PF | 32.8 | VI | 51.7 | QQ | 92.5 | TN | 0.0 |
| AP | 26.3 | PV | 0.0 | TG | 74.0 | AQ | 69.0 | VT | 0.0 |
| NA | 42.0 | FA | 48.8 | VR | 52.5 | AD | 58.3 | EH | 75.4 |
| VH | 47.6 | RL | 30.4 | VQ | 71.2 | SA | 51.0 | SC | 77.7 |
| GL | 63.8 | RW | 34.9 | VS | 0.0 | KM | 84.0 | KV | 71.6 |
| SD | 58.4 | SE | 44.0 | GS | 22.7 | CM | 69.7 | DK | 76.6 |
| RS | 50.3 | SG | 71.4 | SK | 44.9 | TW | 54.8 | LN | 78.8 |
| SQ | 58.8 | WS | 89.0 | TP | 65.7 | ID | 64.6 | EQ | 79.6 |
| RD | 24.1 | DW | 67.3 | TT | 0.0 | ED | 85.1 | DL | 75.8 |
| RF | 0.0 | VV | 89.9 | SV | 31.2 | DS | 84.0 | MD | 64.8 |
| VF | 12.2 | AI | 60.1 | TQ | 51.2 | SW | 0.0 | LH | 81.7 |
| GI | 32.3 | PK | 96.3 | TM | 67.9 | IS | 66.0 | AE | 74.6 |
| QA | 0.0 | ER | 72.8 | RT | 27.5 | CN | 92.0 | DG | 79.7 |
| QI | 0.0 | SS | 93.8 | AG | 40.2 | MQ | 87.5 | FN | 81.4 |
| QG | 0.0 | RR | 90.5 | AL | 48.9 | SF | 0.0 | IQ | 72.0 |
| QP | 0.0 | LK | 82.8 | RY | 67.3 | TA | 0.0 | KY | 72.6 |
| RA | 0.0 | FF | 70.5 | DC | 77.6 | TC | 0.0 | EP | 80.8 |
| HI | 0.0 | FY | 88.0 | DN | 85.6 | TY | 0.0 | PA | 76.8 |
| GP | 0.0 | WG | 91.3 | ES | 48.8 | WT | 0.0 | YI | 44.3 |
| QM | 0.0 | WH | 96.1 | DM | 63.1 | FQ | 0.0 | QW | 42.8 |
| YH | 0.0 | YK | 89.9 | CS | 53.1 | PC | 0.0 | VD | 45.3 |
| HC | 0.0 | RM | 84.7 | AH | 64.7 | PE | 0.0 | LI | 0.0 |
| RI | 0.0 | WP | 68.3 | IY | 68.6 | PL | 0.0 | WC | 18.3 |
| YV | 0.0 | DY | 8.7 | CQ | 93.9 | PM | 0.0 | GD | 23.8 |
| YW | 0.0 | AT | 0.0 | FM | 67.5 | PP | 0.0 | VN | 31.3 |
| YG | 0.0 | WY | 55.6 | QD | 66.0 | AM | 0.0 | GA | 17.6 |
| YP | 0.0 | TI | 36.3 | EL | 56.2 | MV | 0.0 | IL | 3.8 |
| TK | 39.0 | MY | 91.7 | DE | 58.5 | HS | 53.2 | HM | 0.0 |
| QE | 0.0 | AK | 90.3 | VC | 43.3 | VA | 64.7 | VK | 42.0 |
| RN | 10.7 | FK | 73.3 | QT | 25.0 | FI | 45.8 | KC | 20.2 |
| RV | 0.0 | WV | 79.6 | AN | 58.8 | FG | 22.2 | II | 0.0 |
| YN | 0.0 | KD | 90.3 | AS | 72.5 | FH | 39.2 | EF | 13.3 |
| QN | 13.9 | LC | 33.3 | LR | 52.0 | EA | 55.3 | IF | 5.8 |
| WN | 33.1 | HE | 0.0 | AA | 78.4 | GG | 26.1 | TS | 55.7 |
| SH | 51.5 | HV | 0.0 | TD | 77.6 | IA | 38.6 | QL | 0.0 |
| ND | 0.0 | KG | 0.0 | ST | 79.5 | CK | 0.0 | DV | 30.7 |
| HW | 21.6 | GM | 0.0 | SM | 75.1 | TH | 0.0 | GY | 20.7 |
| AY | 31.8 | HK | 43.8 | DD | 64.2 | GE | 0.0 | VE | 24.8 |
| HL | 36.3 | TV | 43.9 | KT | 77.8 | HD | 0.0 | FE | 0.0 |
| FL | 29.2 | YY | 83.5 | NL | 72.6 | CY | 0.0 | DP | 89.3 |
| VP | 50.5 | WA | 88.5 | MT | 71.2 | CV | 0.0 | NK | 0.0 |
| KA | 0.0 | CW | 71.0 | ET | 67.9 | QC | 0.0 | FW | 92.9 |
| CH | 0.0 | PW | 85.5 | IH | 27.1 | EN | 0.0 | WD | 85.9 |
| FV | 0.0 | GF | 83.3 | EI | 74.0 | QR | 33.6 | LE | 92.8 |
| HP | 26.3 | WQ | 88.7 | PQ | 78.1 | DI | 25.9 | SL | 81.6 |
| FP | 0.0 | EG | 90.9 | NV | 80.4 | QH | 14.8 | RP | 88.7 |
| VM | 0.0 | GW | 79.0 | PD | 84.3 | WI | 9.8 | AF | 84.2 |
| GR | 0.0 | DA | 94.1 | IV | 70.9 | AW | 0.0 | RC | 95.1 |
| YL | 24.7 | WR | 83.7 | PI | 87.2 | RH | 0.0 | YD | 88.0 |
| VG | 57.1 | FR | 92.9 | NT | 57.2 | QS | 28.2 | DR | 0.0 |
| HF | 43.2 | TE | 82.5 | NN | 48.3 | GN | 8.5 | CA | 0.0 |
| GV | 1.6 | WK | 78.3 | LS | 0.0 | SR | 52.9 | CR | 0.0 |
| GH | 23.9 | RE | 58.8 | LW | 0.0 | WM | 50.6 | CD | 0.0 |
| QV | 59.7 | YT | 91.5 | LY | 0.0 | CE | 54.4 | CC | 0.0 |
| TL | 0.1 | LV | 79.9 | KN | 0.0 | IP | 59.5 | YQ | 13.5 |
| YF | 12.7 | PG | 80.7 | KR | 0.0 | VL | 47.0 | GQ | 41.4 |
| YS | 0.0 | MW | 76.4 | KQ | 0.0 | TF | 77.3 | QK | 25.9 |
| RQ | 9.7 | MR | 36.5 | LM | 0.0 | EV | 86.4 | QF | 19.0 |
| YC | 5.9 | EE | 86.5 | LP | 0.0 | FC | 0.0 | FT | 0.0 |
| YM | 89.6 | IM | 57.1 | KE | 0.0 | HA | 44.1 | WW | 34.8 |
| NF | 88.2 | EM | 58.4 | KI | 0.0 | FS | 0.0 | SN | 49.9 |
| GK | 87.0 | PS | 71.3 | GT | 0.0 | KH | 8.6 | KS | 0.0 |
| WE | 79.9 | HG | 57.6 | HN | 0.0 | EY | 75.9 | MN | 0.0 |
| LT | 53.4 | KL | 0.0 | HR | 0.0 | EK | 76.5 | ME | 0.0 |
| KW | 76.9 | FD | 13.7 | HQ | 0.0 | DF | 83.4 | MG | 0.0 |
| LA | 47.2 | NS | 31.9 | HT | 0.0 | PH | 74.7 | MH | 0.0 |
| AV | 44.8 | NG | 17.5 | IN | 0.0 | NM | 89.5 | CG | 0.0 |
| WF | 81.7 | PN | 32.7 | IR | 0.0 | PR | 76.4 | CI | 0.0 |
| SP | 94.7 | NR | 72.8 | IC | 0.0 | KK | 0.0 | CL | 0.0 |
| DT | 95.8 | IT | 18.9 | IE | 0.0 | KF | 0.0 | CF | 0.0 |
| SY | 91.5 | PT | 21.7 | IW | 0.0 | KP | 0.0 | CP | 0.0 |
| YR | 83.7 | NY | 58.1 | LD | 0.0 | MM | 0.0 | CT | 0.0 |
| HH | 79.8 | NI | 3.0 | LQ | 0.0 | MF | 0.0 | IG | 67.4 |
| LF | 90.2 | NE | 14.5 | LG | 0.0 | MP | 0.0 | NW | 0.0 |
| WL | 90.2 | NP | 70.6 | LL | 0.0 | MS | 0.0 | RG | 82.8 |
| YA | 80.2 | IK | 71.3 | EC | 0.0 | MI | 0.0 | NQ | 70.1 |
| AR | 95.7 | NC | 83.1 | EW | 0.0 | MC | 0.0 | QY | 54.6 |
| RK | 88.2 | HY | 77.9 | MA | 0.0 | ML | 0.0 | NH | 65.8 |
| VY | 83.2 | AC | 70.8 | GC | 0.0 | MK | 0.0 | DQ | 52.4 |

In Table 1, A=alanine, C=cysteine, D=aspartic acid, E=glutamic acid, F=phenylalanine, G=glycine, H=histidine, I=isoleucine, K=lysine, L=leucine, M=methionine, N=asparagine, P=proline, Q=glutamine, R=arginine, S=serine, T=threonine, V=valine, W=tryptophan, and Y=tyrosine.

As can be seen from the data of Table 1, only some of the 400 dipeptides combined from 20 amino acids exhibited inhibitory activity against PGE₂ production. Only the dipeptides effective for the suppression of PGE₂ production are summarized in Table 2, below.

TABLE 2
Dipeptides Effective for Suppressing PGE₂ Production

| | % | | % | | % | | % | | % | | % |
|---|---|---|---|---|---|---|---|---|---|---|---|
| Sample | Inhibition | Sample | Inhibition | Sample | Inhibition | Sample | Inhibition | Sample | Inhibition | Sample | Inhibition |
| SI | 50.3 | YK | 89.9 | VC | 43.3 | MD | 64.8 | WF | 81.7 | NR | 72.8 |
| YE | 74.2 | RM | 84.7 | QT | 25.0 | LH | 81.7 | SP | 94.7 | IT | 18.9 |
| AP | 26.3 | WP | 68.3 | AN | 58.8 | AE | 74.6 | DT | 95.8 | PT | 21.7 |
| NA | 42.0 | WY | 55.6 | AS | 72.5 | DG | 79.7 | SY | 91.5 | NY | 58.1 |
| VH | 47.6 | TI | 36.3 | LR | 52.0 | FN | 81.4 | YR | 83.7 | NE | 14.5 |
| GL | 63.8 | MY | 91.7 | AA | 78.4 | IQ | 72.0 | HH | 79.8 | NP | 70.6 |
| SD | 58.4 | AK | 90.3 | TD | 77.6 | KY | 72.6 | LF | 90.2 | IK | 71.3 |
| RS | 50.3 | FK | 73.3 | ST | 79.5 | EP | 80.8 | WL | 90.2 | NC | 83.1 |
| SQ | 58.8 | WV | 79.6 | SM | 75.1 | PA | 76.8 | YA | 80.2 | HY | 77.9 |
| RD | 24.1 | KD | 90.3 | DD | 64.2 | YI | 44.3 | AR | 95.7 | AC | 70.8 |
| VF | 12.2 | LC | 33.3 | KT | 77.8 | QW | 42.8 | RK | 88.2 | NL | 72.6 |
| GI | 32.3 | HK | 43.8 | DH | 59.2 | VD | 45.3 | VY | 83.2 | MT | 71.2 |
| TK | 39.0 | TV | 43.9 | QQ | 92.5 | WC | 18.3 | YY | 83.5 | ET | 67.9 |
| RN | 10.7 | TR | 53.0 | AQ | 69.0 | GD | 23.8 | WA | 88.5 | IH | 27.1 |
| QN | 13.9 | VI | 51.7 | AD | 58.3 | VN | 31.3 | CW | 71.0 | EI | 74.0 |
| WN | 33.1 | TG | 74.0 | SA | 51.0 | GA | 17.6 | PW | 85.5 | PQ | 78.1 |
| SH | 51.5 | VR | 52.5 | KM | 84.0 | VK | 42.0 | GF | 83.3 | NV | 80.4 |
| HW | 21.6 | VQ | 71.2 | CM | 69.7 | KC | 20.2 | WQ | 88.7 | PD | 84.3 |
| AY | 31.8 | GS | 22.7 | TW | 54.8 | EF | 13.3 | EG | 90.9 | IV | 70.9 |
| HL | 36.3 | SK | 44.9 | ID | 64.6 | TS | 55.7 | GW | 79.0 | PI | 87.2 |
| PY | 24.8 | TP | 65.7 | ED | 85.1 | DV | 30.7 | DA | 94.1 | NT | 57.2 |
| PF | 32.8 | SV | 31.2 | DS | 84.0 | GY | 20.7 | WR | 83.7 | NN | 48.3 |
| FA | 48.8 | TQ | 51.2 | IS | 66.0 | VE | 24.8 | FR | 92.9 | QR | 33.6 |
| RL | 30.4 | TM | 67.9 | CN | 92.0 | FL | 29.2 | TE | 82.5 | DI | 25.9 |
| RW | 34.9 | RT | 27.5 | MQ | 87.5 | VP | 50.5 | WK | 78.3 | QH | 14.8 |
| SE | 44.0 | AG | 40.2 | HS | 53.2 | HP | 26.3 | RE | 58.8 | QS | 28.2 |
| SG | 71.4 | AL | 48.9 | VA | 64.7 | YL | 24.7 | YT | 91.5 | SR | 52.9 |
| WS | 89.0 | RY | 67.3 | FI | 45.8 | VG | 57.1 | LV | 79.9 | WM | 50.6 |
| DW | 67.3 | DC | 77.6 | FG | 22.2 | HF | 43.2 | PG | 80.7 | CE | 54.4 |
| VV | 89.9 | DN | 85.6 | FH | 39.2 | GH | 23.9 | MW | 76.4 | IP | 59.5 |
| AI | 60.1 | ES | 48.8 | EA | 55.3 | QV | 59.7 | MR | 36.5 | VL | 47.0 |
| PK | 96.3 | DM | 63.1 | GG | 26.1 | YF | 12.7 | EE | 86.5 | TF | 77.3 |
| ER | 72.8 | CS | 53.1 | IA | 38.6 | YM | 89.6 | IM | 57.1 | EV | 86.4 |
| SS | 93.8 | AH | 64.7 | EH | 75.4 | NF | 88.2 | EM | 58.4 | HA | 44.1 |
| RR | 90.5 | IY | 68.6 | SC | 77.7 | GK | 87.0 | PS | 71.3 | EY | 75.9 |
| LK | 82.8 | CQ | 93.9 | KV | 71.6 | WE | 79.9 | HG | 57.6 | EK | 76.5 |
| FF | 70.5 | FM | 67.5 | DK | 76.6 | LT | 53.4 | FD | 13.7 | DF | 83.4 |
| FY | 88.0 | QD | 66.0 | LN | 78.8 | KW | 76.9 | NS | 31.9 | PH | 74.7 |
| WG | 91.3 | EL | 56.2 | EQ | 79.6 | LA | 47.2 | NG | 17.5 | NM | 89.5 |
| WH | 96.1 | DE | 58.5 | DL | 75.8 | AV | 44.8 | PN | 32.7 | PR | 76.4 |
| DP | 89.3 | SL | 81.6 | YD | 88.0 | QF | 19.0 | RG | 82.8 | DQ | 52.4 |
| FW | 92.9 | RP | 88.7 | YQ | 13.5 | WW | 34.8 | NQ | 70.1 | | |
| WD | 85.9 | AF | 84.2 | GQ | 41.4 | SN | 49.9 | QY | 54.6 | | |
| LE | 92.8 | RC | 95.1 | QK | 25.9 | IG | 67.4 | NH | 65.8 | | |

In addition, the compositions comprising the dipeptides of Table 2 may be formulated into a variety of final products.

For example, 1.0 wt % of one or more dipeptides of Table 2, 5.0 wt % of glycerin, 3.0 wt % of 1,3-butylene glycol, 1.0 wt % of PEG-1500, 0.1 wt % of alantoin, 0.3 wt % of DL-panthenol, 0.02 wt % of EDTA-2NA, 0.04 wt % of benzophenon-9, 5.0 wt % of sodium hyaluronate, 10.0 wt % of ethanol, 0.3 wt % of octyldodeceth-16, 0.3wt % of polysorbate-20 and trace amounts of a preservative, a fragrant and a pigment, and a quasi amount of purified water may be mixed to prepare a stringent skin lotion. These contents are given only for illustration, and can vary within broad ranges.

Also, an ointment may be prepared by mixing 1.0 wt % of one or more dipeptides of Table 2, 2.0 wt % of glycerin, 2.0 wt % of 1,3-butylene glycol, 1.0 wt % of PEG-1500, 0.2 wt % of alantoin, 0.2 wt % of DL-panthenol, 3.0 wt % of sodium hyarulonate, 15.0 wt % of ethanol, 0.2 wt % of octyldodeth-16, 0.3 wt % of polysorbate-20, 2.0 wt % of a witch hazel extract, and trace amounts of citric acid, a preservative, a fragrant and a pigment, and a quasi amount of purified water. These contents are given only for illustration, and can vary within broad ranges.

Further, the dipeptides of Table 2 can be used for preparing cosmetics such as skin lotions, lotions, creams, foundations, essences, gels, packs, foam cleansers, soaps, and

### Industrial Applicability

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for suppressing UV-induced erythema, comprising as an active ingredient a dipeptide selected from the group consisting of SI(serine-isoleucine), YK(tyrosine-lysine), VC(valine-cysteine), MD(methionine-aspartic acid), WF(tryptophan-phenylalanine), NR(asparagine-arginine), DP(aspartic acid-proline), YE(tyrosine-glutamic acid), RM(arginine-methionine), QT(glutamine-threonine), LH(leucine-histidine), SP(serine-proline), IT(isoleucine-threonine), FW(phenylalanine-tryptophan), AP(alanine-proline), WP(tryptophan-proline), AN(alanine-asparagine), AE(alanine-glutamic acid), DT(aspartic acid-threonine), PT(proline-threonine), WD(tryptophan-aspartic acid), NA(asparagine-alanine), WY(tryptophan-tyrosine), AS(alanine-serine), DG(aspartic acid-glycine), SY(serine-tyrosine), NY(asparagine-tyrosine), LE(leucine-glutamic acid), VH(valine-histidine), TI(threonine-isoleucine), LR(leucine-arginine), FN(phenylalanine-asparagine), YR(tyrosine-arginine), NE(asparagine-glutamic acid), SL(serine-leucine), GL(glycine-leucine), MY(methionine-tyrosine), AA(alanine-alanine), IQ(isoleucine-glutamine), HH(histidine-histidine), NP(asparagine-proline), RP(arginine-proline), SD(serine-aspartic acid), AK(alanine-lysine), TD(threonine-aspartic acid), KY(lysine-tyrosine), LF(leucine-phenylalanine), IK(isoleucine-lysine), AF(alanine-phenylalanine), RS(arginine-serine), FK(phenylalanine-lysine), ST(serine-threonine), EP(glutamic acid-proline), WL(tryptophan-leucine), NC(asparagine-cysteine), RC(arginine-cysteine), SQ(serine-glutamine), WV(tryptophan-valine), SM(serine-methionine), PA(proline-alanine), YA(tyrosine-alanine), HY(histidine-tyrosine), YD(tyrosine-aspartic acid), RD(arginine-aspartic acid), KD(lysine-aspartic acid), DD(aspartic acid-aspartic acid), YI(tyrosine-isoleucine), AR(alanine-arginine), AC(alanine-cysteine), YQ(tyrosine-glutamine), VF(valine-phenylalanine), LC(leucine-cysteine), KT(lysine-threonine), QW(glutamine-tryptophan), RK(arginine-lysine), NL(asparagine-leucine), GQ(glycine-glutamine), GI(glycine-isoleucine), HK(histidine-lysine), DH(aspartic acid-histidine), VD(valine-aspartic acid), VY(valine-tyrosine), MT(methionine-threonine), QK(glutamine-lysine), TK(threonine-lysine), TV(threonine-valine), QQ(glutamine-glutamine), WC(tryptophan-cysteine), YY(tyrosine-tyrosine), ET(glutamic acid-threonine), QF(glutamine-phenylalanine), RN(arginine-asparagine), TR(threonine-arginine), AQ(alanine-glutamine), GD(glycine-aspartic acid), WA(tryptophan-alanine), IH(isoleucine-histidine), WW(tryptophan-tryptophan), QN(glutamine-asparagine), VI(valine-isoleucine), AD(alanine-aspartic acid), VN(valine-asparagine), CW(cysteine-tryptophan), EI(glutamic acid-isoleucine), SN(serine-asparagine), WN(tryptophan-asparagine), TG(threonine-glycine), SA(serine-alanine), GA(glycine-alanine), PW(proline-tryptophan), PQ(proline-glutamine), IG(isoleucine-glycine), SH(serine-histidine), VR(valine-arginine), KM(lysine-methionine), VK(valine-lysine), GF(glycine-phenylalanine), NV(asparagine-valine), RG(arginine-glycine), HW(histidine-tryptophan), VQ(valine-glutamine), CM(cysteine-methionine), KC(lysine-cysteine), WQ(tryptophan-glutamine), PD(proline-aspartic acid), NQ(asparagine-glutamine), AY(alanine-tyrosine), GS(glycine-serine), TW(threonine-tryptophan), EF(glutamic acid-phenylalanine), EG(glutamic acid-glycine), IV(isoleucine-valine), QY(glutamine-tyrosine), HL(histidine-leucine), SK(serine-lysine), ID(isoleucine-aspartic acid), TS(threonine-serine), GW(glycine-tryptophan), PI(proline-isoleucine), NH(asparagine-histidine), PY(proline-tyrosine), TP(threonine-proline), ED(glutamic acid-aspartic acid), DV(aspartic acid-valine), DA(aspartic acid-alanine), NT(asparagine-threonine), DQ(aspartic acid-glutamine), PF(proline-phenylalanine), SV(serine-valine), DS(aspartic acid-serine), GY(glycine-tyrosine), WR(tryptophan-arginine), NN(asparagine-asparagine), FA(phenylalanine-alanine), TQ(threonine-glutamine), IS(isoleucine-serine), VE(valine-glutamic acid), FR(phenylalanine-arginine), QR(glutamine-arginine), RL(arginine-leucine), TM(threonine-methionine), CN(cysteine-asparagine), FL(phenylalanine-leucine), TE(threonine-glutamic acid), DI(aspartic acid-isoleucine), RW(arginine-tryptophan), RT(arginine-threonine), MQ(methionine-glutamine), VP(valine-proline), WK(tryptophan-lysine), QH(glutamine-histidine), SE(serine-glutamic acid), AG(alanine-glycine), HS(histidine-serine), HP(histidine-proline), RE(arginine-glutamic acid), QS(glutamine-serine), SG(serine-glycine), AL(alanine-leucine), VA(valine-alanine), YL(tyrosine-leucine), YT(tyrosine-threonine), SR(serine-arginine), WS(tryptophan-serine), RY(arginine-tyrosine), FI(phenylalanine-isoleucine), VG(valine-glycine), LV(leucine-valine), WM(tryptophan-methionine), DW(aspartic acid-tryptophan), DC(aspartic acid-cysteine), FG(phenylalanine-glycine), HF(histidine-phenylalanine), PG(proline-glycine), CE(cysteine-glutamic acid), VV(valine-valine), DN(aspartic acid-asparagine), FH(phenylalanine-histidine), GH(glycine-histidine), MW(methionine-tryptophan), IP(isoleucine-proline), AI(alanine-isoleucine), ES(glutamic acid-serine), EA(glutamic acid-alanine), QV(glutamine-valine), MR(methionine-arginine), VL(valine-leucine), PK(proline-lysine), DM(aspartic acid-methionine), GG(glycine-glycine), YF(tyrosine-phenylalanine), EE(glutamic acid-glutamic acid), TF(threonine-phenylalanine), ER(glutamic acid-arginine), CS(cysteine-serine), IA(isoleucine-alanine), YM(tyrosine-methionine), IM(isoleucine-methionine), EV(glutamic acid-valine), SS(serine-serine), AH(alanine-histidine), EH(glutamic acid-histidine), NF(asparagine-phenylalanine), EM(glutamic acid-methionine), HA(histidine-alanine), RR(arginine-arginine), IY(isoleucine-tyrosine), SC(serine-cysteine), GK(glycine-lysine), PS(proline-serine), EY(glutamic acid-tyrosine), LK(leucine-lysine), CQ(cysteine-glutamine), KV(lysine-valine), WE(tryptophan-glutamic acid), HG(histidine-glycine), EK(glutamic acid-lysine), FF(phenylalanine-phenylalanine), FM(phenylalanine-methionine), DK(aspartic acid-lysine), LT(leucine-threonine), FD(phenylalanine-aspartic acid), DF(aspartic acid-phenylalanine), FY(phenylalanine-tyrosine), QD(glutamine-aspartic acid), LN(leucine-asparagine), KW(lysine-tryptophan), NS (asparagine-serine), PH(proline-histidine), WG(tryptophan-glycine), EL(glutamic acid-leucine), EQ(glutamic acid-glutamine), LA(leucine-alanine), NG(asparagine-glycine), NM(asparagine-methionine), WH(tryptophan-histidine), DE(aspartic acid-glutamic acid), DL(aspartic acid-leucine), AV(alanine-valine), PN(proline-asparagine), PR(proline-arginine) and a combination thereof.

2. The composition of claim 1, wherein the dipeptide is used in an amount of from 0.001 to 30 wt % based on a total amount of the composition.

3. The composition of claim 1, further comprising a pharmaceutically acceptable carrier or excipient.

4. The composition of claim 1, being in a formulation form of a cosmetic selected from the group consisting a skin lotion, a lotion, a cream, a foundation, an essence, a gel, a pack, a foam cleanser, and a soap, or a medicament including a topical ointment.
